(19) United States
(12) Patent Application Publication
(10) Pub. No.: US 2018/0104269 A1
(43) Pub. Date: Apr. 19, 2018

(54) COMPOSITION FOR SUPPRESSING MUSCULAR FATTY CHANGE

(71) Applicant: SUNTORY HOLDINGS LIMITED, Osaka (JP)

(72) Inventors: Yuta OTSUKA, Kyoto (JP); Noriyuki KANZAKI, Kyoto (JP); Shuichi MACHIDA, Chiba (JP)

(73) Assignee: SUNTORY HOLDINGS LIMITED, Osaka (JP)

(21) Appl. No.: 15/568,810

(22) PCT Filed: Apr. 22, 2016
(86) PCT No.: PCT/JP2016/062700

§ 371 Date: Oct. 24, 2017

(30) Foreign Application Priority Data
Apr. 27, 2015 (JP) 2015-090580

(51) Int. Cl. A61K 31/7048; A61K 31/353; A61P 21/00; A61P 43/00; C07D 311/30

(52) U.S. Cl. CPC A61K 31/7048; A61K 31/353; C07D 311/30; A61P 43/00; A61P 21/00

(57) ABSTRACT

The object of the invention is to provide a new composition for suppressing fatty degeneration of a muscle, the composition including a component that is safe and suitable for a long term intake. It was found that quercetin or a glycoside thereof suppresses a differentiation process from myosatellite cells into adipocyte-like cells. In the present invention, quercetin or a glycoside thereof is used in a composition for suppressing fatty degeneration of a muscle.

### TECHNICAL FIELD

The present invention relates to a composition for suppressing fatty degeneration of a muscle, the composition comprising quercetin or a glycoside thereof. Further, the present invention also relates to a composition for suppressing fatty degeneration of a muscle, a composition for improving muscle mass, a composition for improving muscle strength, a composition for suppressing muscular atrophy, and a composition for improving motor function, the compositions comprising quercetin or a glycoside thereof and suppressing a differentiation process from myosatellite cells into adipocyte-like cells. Furthermore, the present invention also relates to use of quercetin or a glycoside thereof for suppressing a differentiation process from myosatellite cells into adipocyte-like cells; and a method for suppressing a differentiation process from myosatellite cells into adipocyte-like cells, the method using quercetin or a glycoside thereof.

### BACKGROUND ART

In a super-aging society in a country with a high percentage of aged people in the population, like Japan, many ideas how middle-aged and elderly people may participate in social activities and spend their leisure time have been proposed. On the other hand, problems of the functional disorder of musculoskeletal organs that become apparent with the aging of the population are difficult to handle by simply extending the conventional approach, since there exists a large number of subjects, and exists examples of serious cases and complex cases of multiple diseases. In particular, the deterioration in the musculoskeletal organ function due to the deterioration in the muscle function results in a vicious cycle of an increase in the risk of falling, bone fracture, long-term bedrest, and further deterioration in the musculoskeletal organ function, and is one of the main causes of musculoskeletal ambulation disability symptom and locomotive syndrome. Further, the functional disorder of the musculoskeletal organ is a problem that has to be solved with urgency in a super-aging society since the functional disorder not only lowers the quality of life (QOL) but also causes poor prognosis of a disease by increasing metabolic disorders and frequency of complications of infectious diseases. Currently, a nutritional approach is being studied in addition to kinesiotherapy such as rehabilitation, and components that may enhance the muscular amount and strength are being found (PTL 1 and 2).

It is recently acknowledged that “improvement in muscle quality” as well as “improvement in muscle mass” is important in preventing and ameliorating the functional disorder of the musculoskeletal organ, and studies of the muscle quality are being conducted. A muscle quality is the muscle strength per a muscle mass or a muscle cross section, and is known to deteriorate with age (NPL 1). The main reason for this is that oil droplets in the muscle increase with age, and such increase is thought to cause a decline in the muscle strength such as the grip strength or the strength to bend and extend knees, as well as a deterioration in the body function such as the speed of walking.

The muscle contains myosatellite cells which are multipotent stem cells. The myosatellite cells are normally undifferentiated, but they are activated and multiply when a muscle is damaged or a muscle is growing, and differentiate into myotube cells to form muscle fiber. It is thus possible to stimulate enhancement of muscles and reproduction of muscles by stimulating differentiation from myosatellite cells into myotube cells, and thereby increase muscle mass and muscle strength, and prevent and ameliorate muscular atrophy. For example, PTL 3 and 4 disclose that Rhodiola rosea of the Crassulaceae family, Crataegus cuneata of the Rose family, the Apocynum venetum of the Apocynaceae family or extracts thereof have the effect on stimulating differentiation from myosatellite cells into myotube cells. In addition, PTL 5 discloses that dipeptide or tripeptide having specific amino acid sequences has an effect on stimulating differentiation of myoblasts into muscle cells.

On the other hand, it is known that the myosatellite cells differentiate into not only myotube cells but also adipocyte-like cells, and the latter causes deterioration of the muscle quality, and results in the decline of the muscle strength per a muscle mass or a muscle cross section. Further, it is reported that the proportion of myosatellite cells that differentiate into adipocyte-like cells increase with age (NPL 2). It can thus be thought that suppressing the process in which myosatellite cells differentiate into adipocyte-like cells is an effective means for preventing the deterioration of the muscle quality or for improving the muscle quality. In addition, it is possible to prevent and suppress the decline of muscle mass and muscle strength, and to prevent and suppress muscular atrophy as a result of suppressing a differentiation process from myosatellite cells into adipocyte-like cells.

However, the mechanism of the differentiation of myosatellite cells into adipocyte-like cells differ from the mechanism of the differentiation of myosatellite cells into myotube cells (NPL 3). And, no component has been found so far that suppresses differentiation of myosatellite cells into adipocyte-like cells.

### CITATION LIST

### Patent Literature

PTL 1: Japanese Publication No. 2009-062346
PTL 2: WO 2011/108487
PTL 3: Japanese Publication No. 2014-015429
PTL 4: Japanese Publication No. 2014-015428
PTL 5: WO 2014/092150

### Non Patent Literature

NPL 1: Journal of Applied Physiology, 86(1), 188-194, 1999.
NPL 2: Mechanisms of Ageing and Development, 123(6), 649-661, 2002.
NPL 3: Nature Cell Biology, 12(2), 143-152, 2010.

### SUMMARY OF INVENTION

### Technical Problem

An object of the present invention is to provide a composition for suppressing fatty degeneration of a muscle, the composition comprising a component that can be taken in safely for a long period of time. Further, another object of the present invention is to provide a composition for suppressing fatty degeneration of a muscle, a composition for improving muscle quality, a composition for improving muscle mass, a composition for improving muscle strength, a composition for suppressing muscular atrophy and a composition for improving motor function, the compositions suppress a differentiation process from myosatellite cells into adipocyte-like cells.

### Solution to Problem

The present inventors found after an extensive study that quercetin, which is one kind of polyphenol, or a glycoside thereof has an effect on suppressing fatty degeneration of a muscle. Specifically, they found that quercetin or a glycoside thereof suppresses a differentiation process from myosatellite cells into adipocyte-like cells, and thus completed the invention.

In other words, the present invention relates to the following matters without being limited thereby.

(1) A composition for suppressing fatty degeneration of a muscle, the composition comprising quercetin or a glycoside thereof.
(2) A composition for suppressing fatty degeneration of a muscle, the composition comprising quercetin or a glycoside thereof, and suppressing a differentiation process from myosatellite cells into adipocyte-like cells.
(3) A composition for improving muscle quality, the composition comprising quercetin or a glycoside thereof, and suppressing a differentiation process from myosatellite cells into adipocyte-like cells.
(4) A composition for improving muscle mass, the composition comprising quercetin or a glycoside thereof, and suppressing a differentiation process from myosatellite cells into adipocyte-like cells.
(5) A composition for improving muscle strength, the composition comprising quercetin or a glycoside thereof, and suppressing a differentiation process from myosatellite cells into adipocyte-like cells.
(6) A composition for suppressing muscular atrophy, the composition comprising quercetin or a glycoside thereof, and suppressing a differentiation process from myosatellite cells into adipocyte-like cells.
(7) A composition for improving motor function, the composition comprising quercetin or a glycoside thereof, and suppressing a differentiation process from myosatellite cells into adipocyte-like cells.
(8) The composition according to any one of (1) to (7), which has an effect on suppressing an increase of adipocyte-like cells in a muscle.
(9) The composition according to any one of (1) to (8), which has an effect on suppressing an increase of oil droplets in a muscle.
(10) The composition according to any one of (1) to (9), which has an effect on suppressing an increase in an amount of triglyceride in a muscle.
(11) The composition according to any one of (1) to (10), wherein the composition is an agent.
(12) Use of quercetin or a glycoside thereof for suppressing a differentiation process from myosatellite cells into adipocyte-like cells.
(13) A method for suppressing a differentiation process from myosatellite cells into adipocyte-like cells, the method comprising administering quercetin or a glycoside thereof.

### Advantageous Effects of Invention

The present invention enables using quercetin or a glycoside thereof in compositions for suppressing fatty degeneration of a muscle, improving muscle quality, improving muscle mass, improving muscle strength, suppressing muscular atrophy, and improving motor function. Further, since the composition of the present invention contributes to enhancing the muscle quality, it provides a new means that is beneficial in improving the QOL of people with a disease or of old age.

Further, quercetin or a glycoside thereof has various physiological activities such as an effect of improving blood flow or an anticancer effect. What is more, these components are contained in plants that are edible, so they are quite safe. Accordingly, the quercetin or glycoside thereof of the present invention is expected to provide useful physiological activities other than the effect of suppressing fatty degeneration of a muscle, and they can provide agents that are safe and is susceptible to a continuous intake.

### BRIEF DESCRIPTION OF DRAWINGS

FIG. 1 shows an oil red-O staining image of myosatellite cells derived from a rat after being subjected to adipogenic differentiation culture under the condition of quercetin addition (72 h of culture in adipogenic induction medium→72 h of culture in adipogenic maintenance medium).

FIG. 2 shows an oil red-O staining image of myosatellite cells derived from a rat after being subjected to adipogenic differentiation culture under the condition of quercetin or PPAR γ inhibitor (GW9226) addition (72 h of culture in adipogenic induction medium→72 h of culture in adipogenic maintenance medium).

FIG. 3 shows measurement results of an amount of triglyceride in myosatellite cells derived from a rat after being subjected to adipogenic differentiation culture under the condition of quercetin addition (72 h of culture in adipogenic induction medium→72 h of culture in adipogenic maintenance medium).

### DESCRIPTION OF EMBODIMENTS

The present invention relates to a composition for suppressing fatty degeneration of a muscle, the composition comprising quercetin or a glycoside thereof as an active component. Further, the present invention also relates to a composition for suppressing fatty degeneration of a muscle, a composition for improving muscle quality, a composition for improving muscle mass, a composition for improving muscle strength, a composition for suppressing muscular atrophy, and a composition for improving motor function, the compositions comprising quercetin or a glycoside thereof as an active component, and suppressing a differentiation process from myosatellite cells into adipocyte-like cells. Note that the composition for suppressing fatty degeneration of a muscle, the composition for suppressing fatty degeneration of a muscle and suppressing a differentiation process from myosatellite cells into adipocyte-like cells, the composition for improving muscle quality and suppressing a differentiation process from myosatellite cells into adipocyte-like cells, the composition for improving muscle quantity and suppressing a differentiation process from myosatellite cells into adipocyte-like cells, the composition for improving muscle strength and suppressing a differentiation process from myosatellite cells into adipocyte-like cells, the composition for suppressing muscular atrophy and suppressing a differentiation process from myosatellite cells into adipocyte-like cells, and the composition for improving motor function and suppressing a differentiation process from myosatellite cells into adipocyte-like cells may be collectively referred to as the “composition for suppressing fatty degeneration of a muscle, etc.” in the present specification.

“Fatty degeneration of a muscle” as used in the present specification refers to the differentiation of myosatellite cells into adipocyte-like cells. Accordingly, “suppressing fatty degeneration of a muscle” as used in the present specification refers to suppressing a differentiation process from myosatellite cells into adipocyte-like cells. The fatty degeneration of a muscle may be assessed by measuring the amount of oil droplet in the muscle tissue by measuring the amount of oil red-O stain of the muscle tissue or triglyceride in the muscle tissue.

Further, the “muscle quality” as used in the present specification refers to the muscle strength per a muscle mass or a muscle cross section. Accordingly, “improving muscle quality” as used in the present specification refers to increasing muscle strength more than increasing muscle mass or muscle cross section. Note that “improving muscle mass” refers to preventing or suppressing the reduction in the number of muscle fibers in the muscle tissue or the reduction in the cross sectional area of muscle fibers, or to preventing or suppressing the reduction of the muscle protein synthesis speed compared to the muscle protein decomposition speed in the muscle tissue. Further, “improving muscle strength” refers to preventing or suppressing the decline in the potential tension of each muscle, and may be measured by the strength to bend and extend knees or the grip strength.

Furthermore, “muscular atrophy” as used in the present specification refers to the reduction in muscle mass through the decrease in the number or the size of the myocytes due to the inclination of the metabolic turnover of muscle synthesis and muscle decomposition towards decomposition. Muscular atrophy may be roughly categorized into that from long term bedrest, casting due to bone fracture, etc., illness, and aging, etc. Accordingly, “suppressing muscular atrophy” leads to suppressing the deterioration of the motor function or the reduction of muscle mass that occurs due to the above reasons.

### 1. Quercetin and Quercetin Glycoside

An aspect of the present invention is a composition for suppressing fatty degeneration of a muscle comprising quercetin or a glycoside thereof as an active component. “Quercetin” as used in the present specification refers to quercetin, which is a type of polyphenol, and is represented by Formula (I).

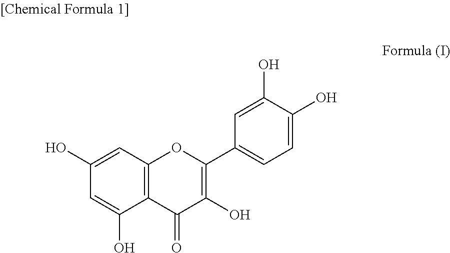 [SMILES: O=c1c(O)c(-c2ccc(O)c(O)c2)oc2cc(O)cc(O)c12]

The composition for suppressing fatty degeneration of a muscle, etc. of the present invention may comprise quercetin glycoside as an active component. Quercetin glycoside is a compound in which a glycosidic bond is formed between quercetin and one or more sugar chains, and specifically, a series of compounds in which a glycosidic bond is formed between a hydroxyl group at position 3 and one or more sugar chains. Quercetin and quercetin glycoside are quite different in terms of chemical structure as well as chemical properties. Since quercetin is insoluble to water, it is mostly taken in from plants, etc. as glycoside to be absorbed in the body through the digestive tract before it is turned into quercetin by the work of the digestive enzyme or the metabolic enzyme so as to provide desired effects in the body.

The “quercetin glycoside” as used in the present specification is represented by formula (II).

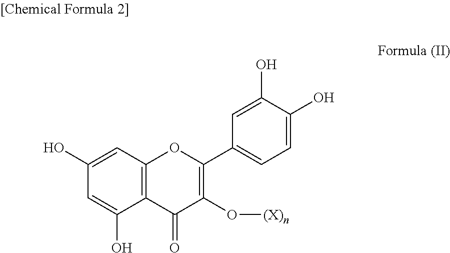

wherein, (X)n is a sugar chain, and n is an integer that is 1 or higher.

Here, the sugar, represented by X, that constitutes a sugar chain that binds with quercetin to form a glycosidic bond includes glucose, rhamnose, galactose, glucuronic acid, etc., and is preferably glucose or rhamnose. Further, there is no limitation to the value of n as long as it is 1 or higher, but values of 1 to 16 are preferred, and values of 1 to 8 are even more preferred. When the value of n is 2 or higher, the X section may consist of a single type of sugar chain or of multiple types of sugar chains.

The quercetin glycoside of the present invention includes quercetin glycosides obtained by treating an existing quercetin with an enzyme, etc. to cause a sugar transfer. The quercetin glycoside as mentioned in the present invention specifically includes rutin, an enzyme-treated rutin, quercitrin, and isoquercitrin.

In a particularly preferable aspect of the present invention, the enzyme-treated rutin is used as the quercetin glycoside. A preferable examples of enzyme-treated rutin include isoquercitrin obtained by treating quercetin glycoside with an enzyme to remove the rhamnose sugar chain section, and a compound obtained by treating isoquercitrin with a glycosyltransferase to bind isoquercitrin with a sugar chain consisting of 1 to 7 glucoses, or mixtures thereof as the main components.

The present invention may use quercetin or a glycoside thereof alone, or in a mixture of multiple compounds. The quercetin or a glycoside thereof to be used in the present invention is not limited by its origin or production process, and may be obtained, for example, from a plant that contains a substantial amount of quercetin or glycoside thereof. For example, buckwheat, Styphnolobium japonicum, caper, apple, tea, onion, grape, broccoli, Corchorus olitorius, raspberry, blueberry, cranberry, Opuntia, leaf vegetables, and citrus fruits are known as plants containing a substantial amount of quercetin or glycosides thereof, and quercetin or glycosides thereof may be obtained from these plants. The quercetin or a quercetin thereof used in the present invention may be those with a high quercetin or quercetin glycoside content obtained by concentrating or purifying quercetin or a glycoside thereof extracted from natural products, such as concentrates or purified products of extracts containing quercetin or a glycoside thereof. When concentrating or purifying quercetin or a glycoside thereof, known methods may be used.

### 2. Composition for Suppressing Fatty Degeneration of a Muscle, Etc.

The present invention relates to a composition for suppressing fatty degeneration of a muscle comprising quercetin or a glycoside thereof; a composition for suppressing fatty degeneration of a muscle comprising quercetin or a glycoside thereof and suppressing a differentiation process from myosatellite cells into adipocyte-like cells; a composition for improving muscle quantity comprising quercetin or a glycoside thereof and suppressing a differentiation process from myosatellite cells into adipocyte-like cells; a composition for improving muscle strength comprising quercetin or a glycoside thereof and suppressing a differentiation process from myosatellite cells into adipocyte-like cells; a composition for suppressing muscular atrophy comprising quercetin or a glycoside thereof and suppressing a differentiation process from myosatellite cells into adipocyte-like cells; and a composition for improving motor function comprising quercetin or a glycoside thereof and suppressing a differentiation process from myosatellite cells into adipocyte-like cells (composition for suppressing fatty degeneration of a muscle, etc.).

The content of quercetin or a glycoside thereof in the composition for suppressing fatty degeneration of a muscle, etc. of the present invention is not particularly limited, and may be any amount that provides the desired effect of the present invention in view of the form of administration and the administration method. For example, the content of quercetin or a glycoside thereof in the composition for suppressing fatty degeneration of a muscle, etc. of the present invention is 0.1 mg to 8000 mg, preferably 0.3 mg to 4000 mg, more preferably 1.0 mg to 1000 mg, and even more preferably 10 mg to 100 mg, as calculated in terms of quercetin content. Further, the weight percent of quercetin or a glycoside thereof in the composition for suppressing fatty degeneration of a muscle, etc. of the present invention is not particularly limited, but may be 0.001 to 95 wt %, preferably 0.01 to 80 wt %, more preferably 0.1 to 50 wt %, and even more preferably 1.0 to 20 wt % based on the total weight of the composition.

The content of quercetin or a glycoside thereof may be measured according to known methods, such as HPLC.

### 2-1. Working Mechanism

The muscle tissue contains myosatellite cells, which are multipotent stem cells, and they are normally undifferentiated, but they multiply by activation when the muscle is damaged or when the muscle is growing, and differentiate into myotube cells to form muscle fibers. Thus, promotion of differentiation from myosatellite cells into myotube cells leads to enhancement of muscles, etc. On the other hand, the myosatellite cells differentiate into not only myotube cells but also adipocyte-like cells so that the muscle quality deteriorates and the muscle strength per muscle mass or muscle cross sectional area decreases. Accordingly, suppression of the process in which myosatellite cells differentiate into adipocyte-like cells leads to suppression of fatty degeneration of a muscle. Further, suppression of a differentiation process from myosatellite cells into adipocyte-like cells leads to prevention, suppression or alleviation of deterioration of muscle quality or decrease in muscle mass, decline in muscle strength, and muscular atrophy. In addition to the above, an effect on improving motor function is provided by the above effects of the composition for suppressing fatty degeneration of a muscle, etc. of the present invention.

The “myosatellite cells” as used in the present specification refer to multipotent stem cells present between the basement membrane and the cell membrane of muscle fibers. Accordingly, the myosatellite cells whose differentiation into adipocyte-like cells is suppressed by the present invention may be multipotent stem cells.

The myosatellite cells differentiate into adipocyte-like cells under a specific environment. For example, myosatellite cells differentiate into adipocyte-like cells by being subjected to specific mediums such as adipogenic differentiation inducing medium and adipogenic differentiation stimulating medium. Differentiation of myosatellite cells into adipocyte-like cells are known to increase with age.

The “adipocyte-like cells” as used in the present specification refer to cells with increased oil droplets in the cell. In other words, “adipocyte-like cells” are cells with an increased triglyceride content compared to that in myocytes. In the present specification, the adipocyte-like cells are produced by differentiation of myosatellite cells. An increase in the adipocyte-like cells in the muscle tissue by differentiation of myosatellite cells causes deterioration of muscle quality, decrease in muscle mass, decline of muscle strength, and muscular atrophy, and consequently, a deterioration of motor function.

### 2-2. Other Components

The composition for suppressing fatty degeneration of a muscle, etc. of the present invention may comprise any additive or any component used in general compositions other than quercetin or a glycoside thereof according to the form of the composition. Examples of such additives or components include vitamins such as vitamin A, vitamin B, vitamin E, and vitamin C; minerals; nutritional components; bioactive components; and also agents added to pharmaceutical formulations, such as excipients, binders, emulsifiers, tonicity agents, buffering agents, solubilizers, antiseptics, stabilizers, antioxidants, coloring agents, coagulants, coating agents, and flavors, etc.

### 2-3. Usage

The composition for suppressing fatty degeneration of a muscle, etc. of the present invention may suppress a differentiation process from myosatellite cells into adipocyte-like cells by containing quercetin or a glycoside thereof at an effective dose. This suppresses the increase of adipocyte-like cells, which in turn suppresses the increase of oil droplets in the muscle and the increase of the triglyceride amount in the muscle. Further, when the composition for suppressing fatty degeneration of a muscle, etc. of the present invention is taken in, the process in which myosatellite cells differentiate into adipocyte-like cells is suppressed, so that the differentiation of myosatellite cells into myotube cells is relatively stimulated, thereby bringing about the effect on preventing or suppressing the decrease of muscle mass or the decline of muscle strength and the effect on suppressing muscular atrophy. Further, when the composition for suppressing fatty degeneration of a muscle, etc. of the present invention is taken in, the effect on suppressing the increase in the adipocyte-like cells, the effect on improving the muscle quality, the effect on suppressing the decrease in the muscle mass and the muscle strength, and the effect on suppressing the muscular atrophy are produced, whereby the effect on improving motor functions can be obtained. Furthermore, the present invention may be used for the prevention, amelioration or treatment of the functional disorder of the musculoskeletal organs resulting from the above effects. Examples include the prevention, amelioration or treatment of the functional disorder of the musculoskeletal organs or the locomotive syndrome, etc. resulting from long term bedrest, casting due to bone fracture, etc., illness, aging, etc., without being limited thereby.

The composition for suppressing fatty degeneration of a muscle, etc. of the present invention may be applied to either a therapeutic usage (medical usage) or a non-therapeutic usage (non-medical usage). Specifically, it includes a usage as medicines, etc. Note that “non-therapeutic” is a concept that does not include a medical activity, that is, an activity of treating the human body in therapy.

The composition for suppressing fatty degeneration of a muscle, etc. of the present invention may be provided as an agent, but is not limited to. The agent may be provided as a composition by itself, or as a composition containing the agent. For example, the agent may be provided in the form of a medical composition, a food/beverage composition, a cosmetic composition, without being limited thereby. Further, the present invention may be provided in the form of medicines for animals. Non-limiting examples of food/beverage compositions include functional foods, health supplementary foods, nutritional functional foods, special use foods, food for specified health use, nutritional supplements, foods for dietary cure, health foods, dietary supplements, food additives, etc. Further, the present invention may be provided as a composition which has an indication that explicitly or implicitly displays the effect produced by the composition for suppressing fatty degeneration of a muscle, etc. of the present invention, for example, the effect on suppressing fatty degeneration of a muscle, the effect on improving muscle quality, the effect on improving muscle mass, the effect on improving muscle strength, the effect on suppressing muscular atrophy, and the effect on improving motor function.

The composition for suppressing fatty degeneration of a muscle, etc. of the present invention may be administered by a suitable process according to its form. For example, the dosage form of the composition for suppressing fatty degeneration of a muscle, etc. of the present invention may be oral administration or in the form of injection, etc., and a form known for the formulation suited for each administration may be used. For example, the dosage form includes oral solid formulations such as tablets, coated tablets, pellets, powders, or capsules; oral liquid formulations such as liquid agents for internal use or syrup agents; non-oral formulations such as injections, external preparations, suppositories, or dermal absorbent, without being limited thereby.

When administered to animals, the total amount of quercetin or a glycoside thereof to be administered, as calculated in terms of quercetin amount, may be 0.1 mg to 16 mg, preferably 0.3 mg to 4 mg, more preferably 0.5 mg to 2 mg (per one mouse with about 20 g of body weight). When administered to humans (adults), the total amount of quercetin or a glycoside thereof to be administered, as calculated in terms of quercetin amount, may be 0.1 mg to 8000 mg, preferably 0.3 mg to 4000 mg, more preferably 1.0 mg to 1000 mg, and even more preferably 10 mg to 100 mg.

Further, in the composition for suppressing fatty degeneration of a muscle, etc. of the present invention, the amount of the quercetin or a glycoside thereof contained therein should preferably be an amount that provides the desired effect of the present invention, that is, the effective amount, in view of the dosage form and the administration method. For example, the daily intake of enzyme treated rutin per individual is 0.1 to 20 g, preferably 0.3 to 10 g, and more preferably 0.5 to 5 g. Further, the daily intake of quercetin or a glycoside thereof in the composition for suppressing fatty degeneration of a muscle, etc. of the present invention per 1 kg of body weight is not particularly limited; but, for example, the daily intake of enzyme-treated rutin per 1 kg of body weight is 0.002 to 400 mg, preferably 0.006 to 200 mg, more preferably 0.01 to 50 mg, and even more preferably 0.1 to 10 mg.

3. Use of Quercetin or a Glycoside Thereof to Suppress the Process in which Myosatellite Cells Differentiate into Adipocyte-Like Cells

One aspect of the present invention is the use of quercetin or a glycoside thereof for suppressing the process in which myosatellite cells differentiate into adipocyte-like cells. The above use includes for example use for preventing, alleviating or treating various diseases or symptoms relating to the functional disorder of the musculoskeletal organs, including musculoskeletal ambulation disability symptom or locomotive syndrome, etc. resulting from long term bedrest, casting due to bone fracture, etc., illness, and aging, etc., without being limited thereby. Further, the use is a use in human or non-human animal, and it may be a therapeutic usage or a non-therapeutic usage.

4. A Method for Suppressing a Differentiation Process from Myosatellite Cells into Adipocyte-Like Cells

An aspect of the present invention provides a method for suppressing a differentiation process from myosatellite cells into adipocyte-like cells, the method comprising administering quercetin or a glycoside thereof to a subject. The dosage of the present invention is not particularly limited as long as it is an amount that provides the above desired effect, that is, the effective amount. Furthermore, the method of the present invention enables suppression of the differentiation of myosatellite cells into adipocyte-like cells without causing side-effects. Note that the subject to be administered the quercetin or a glycoside thereof in the present invention method is the same as the subject to be administered the agent of the present invention.

The effective amount in the present invention is an amount that suppresses the differentiation of myosatellite cells into adipocyte-like cells when the quercetin or a glycoside thereof of the present invention is administered to the above subject, as compared to the subject that had not been administered the same composition. Specific effective amounts are appropriately set based on the dosage form, the administration method, the purpose of use and the age, body weight, symptom, etc. of the subject.

In the method of the present invention, the quercetin or a glycoside thereof may be administered by itself or may be administered as a composition containing quercetin or a glycoside thereof to satisfy the therapeutic effective amount.

### EXAMPLES

The present invention is described more specifically below, but this does not limit the present invention. A person skilled in the art may use the method of the present invention by changing it or modifying it in various ways, and such variations are also included in the scope of the present invention.

### Example 1: Effect of Quercetin to Suppress Storage of Fat

### (1) Isolation of Myosatellite Cells

The gastrocnemial muscle, the soleus, the anterior tibial muscle, the extensor digitorum longus muscle, and the quadriceps femoris muscle were extracted from the Fischer 344 rat of 10 to 14 weeks old under anesthesia, and minced. The above tissues were subjected to enzymolysis with Protease (Sigma-Aldrich) and subjected to centrifugal treatment to obtain a precipitate. The obtained precipitate was suspended in Dulbecco's Modified Eagle Medium (DMEM) containing 10% horse fetus serum, and seeded on an 8-well slide glass (BD Thermo Science) so that the myosatellite cells are 5×10⁴ to 7×10⁴ cells/mL.

### (2) Induction of Differentiation to Adipocyte-Like Cells

Myosatellite cells isolated by F-10 containing 20% cow serum (GIBCO) was cultured for 72 h. Then, the cells were cultured for 72 h. in an adipogenic induction medium (Lonza) containing 0.1% DMSO (Control), 5 μM quercetin, 10 μM quercetin or 50 μM quercetin. Then, the cells were cultured for 72 h. in an adipogenic maintenance medium (Lonza) containing the control, 5 μM quercetin, 10 μM quercetin or 50 μM quercetin.

Further, an experiment for comparing quercetin with PPARγ inhibitor (GW9662:IC50=3.3 nM) was performed, in which the cells were cultured as shown above under the condition in which 10 μM quercetin, or 1, 5, or 10 μM of GW9662 was added.

### (3) Staining of Oil Droplets in Cells

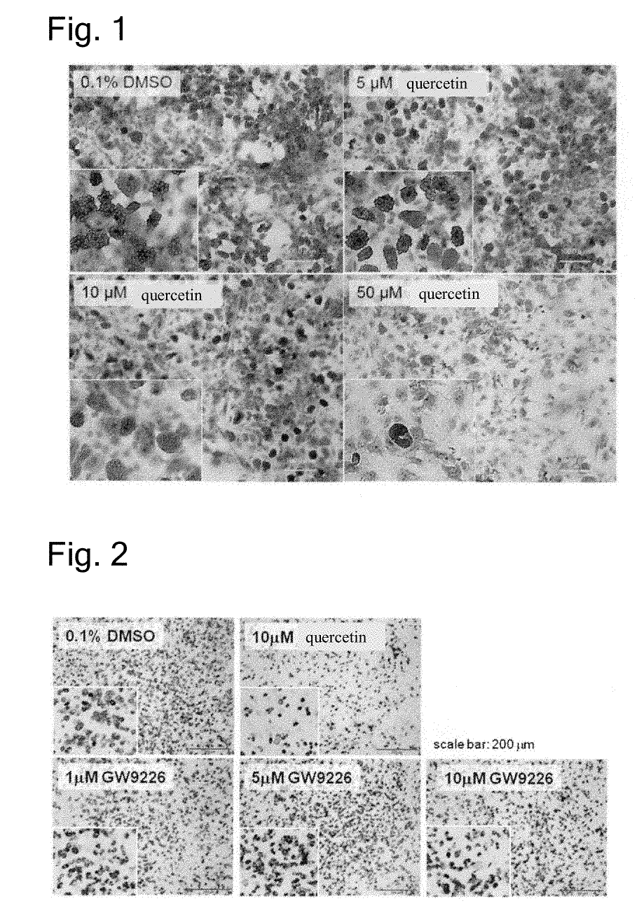

After the cultured cells were washed in PBS, they were fixed by 10% formalin (Wako Pure Chemical Industries, Ltd.) at room temperature for 10 min. Then, they were reacted with 10% oil red-O solution (Sigma Aldrich) that was dissolved in 2-propanol at room temperature for 30 min. Subsequently, the cells were observed with a microscope and the number of adipocyte-like cells stained by oil red-O was measured. The results are shown in FIGS. 1 and 2.

### (4) Quantification of Amount of Triglyceride in Cells

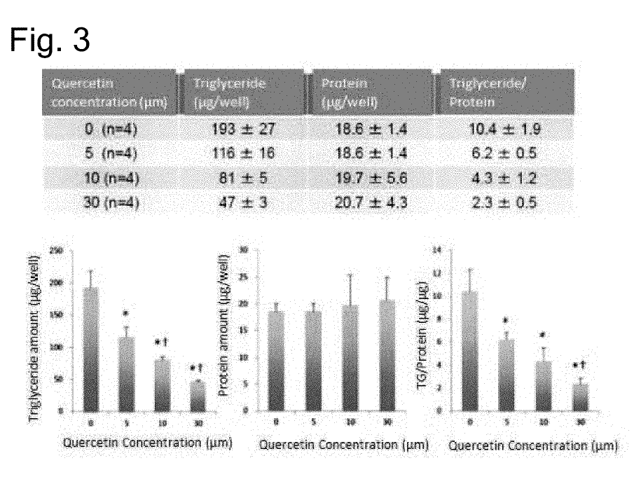

After the cells that were subjected to adipogenic differentiation culture were washed with PBS, a PBS that contains 5% Triton-X 100 was added as a cell lysate, and the cell solution was collected, heated and cooled. After the cells were centrifuged, triglyceride and protein in the supernatant were quantified. The LabAssay™ Triglyceride (Wako Pure Chemical Industries, Ltd.) was used for the quantification of triglyceride. The Pierce BCA Protein Assay Kit (Thermo Scientific) was used for the quantification of protein. FIG. 3 shows the average value±standard error and graphs of the amount of triglyceride (left), the amount of protein (center), the rate of triglyceride/protein in the cells that were subjected to adipogenic differentiation under the presence of respective concentrations of quercetin. The results were tested using the Dunnett's test, and the value of 5% or lower was determined to be the significant amount (*: p<0.05 vs 0 μM. †: p<0.05 vs 5 μM).

As a result of oil red-O staining, adipocyte-like cells with increased oil droplets stained by oil red-O were observed in the control group. When 5 μM quercetin was added, the same level of oil droplets as the control were stained by oil red-O, and no significant decrease in the number of adipocyte-like cells was acknowledged. On the other hand, when 10 μM and 50 μM of quercetin were added, the number of oil droplets that were stained by oil red-O decreased in a concentration dependent manner, and the number of adipocyte-like cells decreased. Note that the number of adipocyte-like cells did not decrease in any of the concentrations, 1, 5, 10 μM of GW9662, so the effect of quercetin to decrease the number of cells is independent of PPARγ.

Further, as a result of quantifying the amount of triglyceride in cells after adipogenic differentiation, it was found that the amount of triglyceride decreased in a quercetin-concentration dependent manner. Further, the amount of protein in the cells in the well did not change by adding quercetin, and the amount of triglyceride corrected by the amount of protein decreased in a quercetin concentration dependent manner. It was thus shown that quercetin suppresses differentiation of myosatellite cells into adipocyte-like cells.

### INDUSTRIAL APPLICABILITY

In the present invention, by administering quercetin or a glycoside thereof as an effective component, a differentiation process from myosatellite cells into adipocyte-like cells is suppressed, and thus fatty degeneration of a muscle is suppressed. Further, quercetin or a glycoside thereof is a component that is contained in edible plants, so it is quite safe, and there is little possibility that an unexpected harm would occur by intake of quercetin or a glycoside thereof. Accordingly, the agent of the present invention is safe and is suitable for a continuous intake, and it has a high industrial applicability as a new means for the prevention, amelioration or treatment of the functional disorder of the musculoskeletal organ resulting from the decreased muscle mass or muscle strength that comes with fatty degeneration of a muscle.

1. A composition for suppressing fatty degeneration of a muscle, the composition comprising quercetin or a glycoside thereof.

2. A composition for suppressing fatty degeneration of a muscle, the composition comprising quercetin or a glycoside thereof, and suppressing a differentiation process from myosatellite cells into adipocyte-like cells.

3. A composition for improving muscle quality, the composition comprising quercetin or a glycoside thereof, and suppressing differentiation process from myosatellite cells into adipocyte-like cells.

4. A composition for improving muscle mass, the composition comprising quercetin or a glycoside thereof, and suppressing a differentiation process from myosatellite cells into adipocyte-like cells.

5. A composition for improving muscle strength, the composition comprising quercetin or a glycoside thereof, and suppressing a differentiation process from myosatellite cells into adipocyte-like cells.

6. A composition for suppressing muscular atrophy, the composition comprising quercetin or a glycoside thereof, and suppressing a differentiation process from myosatellite cells into adipocyte-like cells.

7. A composition for improving motor function, the composition comprising quercetin or a glycoside thereof, and suppressing a differentiation process from myosatellite cells into adipocyte-like cells.

8. The composition according to claim 1, which has an effect on suppressing an increase of adipocyte-like cells in a muscle.

9. The composition according to claim 1, which has an effect on suppressing an increase of oil droplets in a muscle.

10. The composition according to claim 1, which has an effect on suppressing an increase in an amount of triglyceride in a muscle.

11. The composition according to claim 1, wherein the composition is an agent.

12. Use of quercetin or a glycoside thereof for suppressing a differentiation process from myosatellite cells into adipocyte-like cells.

13. A method for suppressing a differentiation process from myosatellite cells into adipocyte-like cells, the method comprising administering quercetin or a glycoside thereof.
